(19) United States
(12) Patent Application Publication
(10) Pub. No.: US 2017/0258692 A1
(43) Pub. Date: Sep. 14, 2017

(54) KERATIN FIBRE DYEING COMPOSITION

(71) Applicant: BEAUTY & BUSINESS S.p.A., Milano (IT)

(72) Inventors: Antonio Consoli, Urgnano (BG) (IT); Katiuscia Grevalcuore, Bergamo (IT); Massimo Fabbi, Mozzo (IT); Emanuela Facchetti, Romano di Lombardia (IT)

(21) Appl. No.: 15/452,915

(22) Filed: Mar. 8, 2017

(30) Foreign Application Priority Data
Mar. 11, 2016 (IT) 102016000025988

(51) Int. Cl. A61K 8/22; A61Q 5/10; A61K 8/34; A61K 8/49; A61K 8/41

(52) U.S. Cl. CPC A61K 8/22; A61K 8/4953; A61K 8/411; A61K 8/347; A61K 8/415; A61K 8/4946; A61K 8/4926; A61Q 5/10; A61K 2800/4324

(57) ABSTRACT

Disclosed is a hair colouring method which comprises application to the hair of at least one dye of formula I and/or II combined with a coupler of formula III:

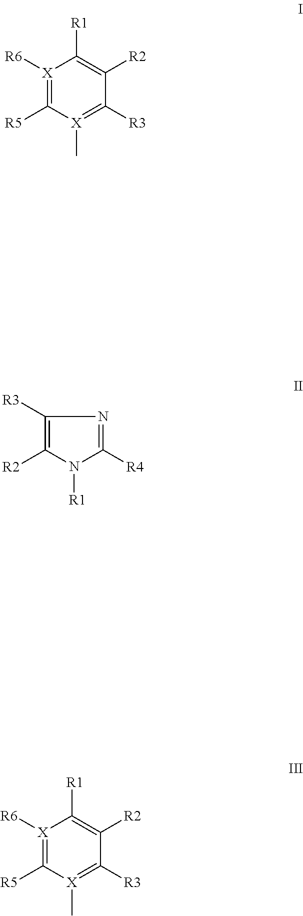

wherein R1-R6 and X are as defined in the description.

This U.S. Non-Provisional Application claims priority to and the benefit from Italian Patent Application No. 102016000025988 filed on Mar. 11, 2016, the content of which is incorporated herein by reference in its entirety.

The invention relates to a hair colouring method comprising the application of primary dyes suitably combined with couplers.

### PRIOR ART

There are currently numerous trends in hair colouring. In the past, the hair was mainly dyed to conceal areas of grey hair, whereas there is now increased demand for fashionable hair colors. Two hair colouring technologies are widely used. The first involves the use of oxidative dyes and an activator (oxidative hair dyes), while the second involves the use of direct dyes.

Oxidative dyes have become very important in the conventional hair colouring field. The color is created by the reaction of primary intermediates and couplers in the presence of an oxidant. Oxidative dyes give the best performance in terms of resistance to washing, and are therefore called permanent hair dyes.

Hair colouring preparations involving the use of direct dyes have become widely used in recent years due to their ability to create particularly bright shades which are unobtainable with oxidative dyes. In addition to natural colors like black, brown, gold and copper, they also enable the user to obtain “crazy” or non-natural colors like violet, red, blue, orange, yellow and green (such as PANTONE red 711XGC, PANTONE yellow 13-0630 TN, PANTONE green 3405C, PANTONE blue PQ-3591 C, PANTONE orange 151 XGC, PANTONE violet PQ-2592C and PANTONE 266 C). “Crazy” colors means bright colors with an intense tone but no level, which give the hair an unnatural color.

Direct dyes can be classified as anionic, cationic and non-ionic.

The hair tints that can be created with direct dyes can be classified, according to their ability to resist washing, as semipermanent or temporary. Semipermanent hair dyes can resist up to 8 washes, and temporary dyes up to 2 washes (see Table A).

TABLE A

| | TEM- | SEMI- | SEMI- | PER- |
|---|---|---|---|---|
| | PORARY | PER- | PERMANENT | MANENT |
| | (direct | MANENT | (oxidation | (oxidation |
| | dyes) | (direct dyes) | dyes) | dyes) |
| DURABILITY | 1-2 | 6-8 | 6-8 weeks | Permanent |
| PROPERTIES | shampoos | shampoos | | |
| LIGHTENING | No | No | No | Yes |
| EFFECT | | | | |
| COVERING | No | YES | YES | YES |
| GREY HAIR | | (maximum | (maximum | (100% |
| | | 30% grey | 50% grey hair) | grey hair) |
| | | hair | | |
| BLEACHING | YES | preferably | Unnecessary | Un- |
| PRE- | | | | necessary |
| TREATMENT | | | | |
| COLOUR | Natural and | Natural and | Natural colors | Natural |
| RESULT | crazy colors | crazy colors | | colors |
| FINISHED | Ready to | Ready to | Mix before | Mix before |
| PRODUCT | use | use | use with | use with |
| | | | activator | activator |
| | | | (e.g. hydrogen | (e.g. |
| | | | peroxide). | hydrogen |
| | | | | peroxide) |

Cationic or basic dyes have been widely used in recent years. These dyes bind to the keratin fibre much more efficiently than anionic or non-ionic dyes. Cationic dyes are more resistant to washing than the other two categories, and can therefore also be used to create semipermanent hair dyes.

Two particularly important cationic dyes, called Basic Red 51 and Basic Yellow 87 according to the INCI nomenclature (European Community Decision 2006/257/EC as amended—International Nomenclature of Cosmetic Ingredients), were introduced onto the market in the 1990s. EP 0681464 reports the chemical structures of Basic Red 51 (BR51) and Basic Yellow 87 (BY87). Basic Red 51 and Basic Yellow 87 can be combined to give shades ranging from intense red to orange.

Basic Blue 124 (BB124) was introduced by BASF in 2013 (U.S. Pat. No. 8,268,014), thus completing the series of three cationic primary colors with which each type of shade (from natural to “crazy” colors) can be created.

BB124 and BY87 can be combined in a formulation to obtain greens, BB124 and BR51 to obtain violets, and BB124, BY87 and BR51 to obtain colors ranging from black through chestnut to blonde with a natural tone. These dyes give the keratin fibre a very bright tone, but although they are the best performing hair dyes, their resistance to washing is limited to a semipermanent type of technology.

A particularly strongly felt problem is that of obtaining “crazy” colors which are very bright, but permanent.

The hair tints available to hairdressers consist of combinations of dyes defined as secondary colors, which are usually called shades. The International Colour Chart (ICC) is a hair tints classification system. This means that every hair colouring composition has a code defining its color result. Said code can be used by manufacturers of color charts or dyes. In practice, the ICC system uses numbers to define the depth (level) and tone of a given color.

The color level indicates how light or dark the shade is. The ICC system assigns a number to measure the level of lightness or darkness. Said values range from 1 to 10, wherein 1 denotes the darkest shade (black) and 10 the lightest shade (lightest blonde). Some manufacturers also use the codes 11 and 12 on their hair colouring products to indicate platinum blonde shades. The usual level numbers and names are as follows:

TABLE B

| Level | Level name |
|---|---|
| 1 | Black |
| 2 | Very dark brown |
| 3 | Dark brown |
| 4 | Medium brown |
| 5 | Light brown |
| 6 | Dark blonde |
| 7 | Medium blonde |
| 8 | Light blonde |
| 9 | Very light blonde |
| 10 | Lightest blonde |
| 11 | Platinum blonde |

The tone indicates how cool or warm a color is, and includes colors such as gold, ash and copper.

Although the level measurement is almost identical for all manufacturers, each manufacturer can vary the tone number at its discretion. Tone is indicated by a number, usually placed after the level, separated by a decimal point “.”, a comma “,” or a slash “/”. The classification used by the Applicant is set out below by way of example

TABLE C

| Tone number | Tone name |
|---|---|
| 0 | Natural (grey-neutral) |
| 1 | Ash (blue) |
| 2 | Irise (violet) |
| 3 | Gold (yellow) |
| 4 | Copper (orange) |
| 5 | Mahogany (violet red) |
| 6 | Red (red) |
| 7 | Matte (green) |
| 8 | Pearl |

Some hair tints can have a double tone, and it is usual to place two numbers after the decimal point of the level to express said characteristic. For example, if the color chart contains the number 7.21, the first number indicates the medium blonde level (7), the second indicates the irisé tone (2), and the third number indicates a secondary blue ash tone (1). Said color will be called “medium blonde irisé ash”. When the two numbers indicating the tone are the same, the tone is intense. For example, 7.44 indicates a medium blonde intense copper. Colours which have no level but only an intense, bright tone are often defined as “crazy” colors, and this definition will be used in the description of the present invention. Examples are blue, red, orange, yellow, green and violet (e.g.: PANTONE red 711XGC, PANTONE yellow 13-0630 TN, PANTONE green 3405C, PANTONE blue PQ-3591 C, PANTONE orange 151 XGC, PANTONE violet PQ-2592C and PANTONE 266 C).

When a direct dye is used to obtain “crazy” colors with bright tones, it is unfortunately necessary to perform a pre-treatment to bleach the hair at least to level 7 or 8, and preferably to level 10, to obtain a result not influenced by the melanin residue. The direct dye is then applied to the bleached hair. The melanin residue is the type and quantity of melanin that remains in the keratin fibre after the hair is bleached (the color ranges between brown-orange and yellow).

If a direct dye is used on level 1 to 6 hair without a bleaching pre-treatment, it is very difficult to obtain an intense, bright color, and the result is a light secondary tone.

Another limitation on the use of direct dyes is the unevenness of the result which, depending on the hair type (normal or damaged/treated), can give different results in terms of tone and resistance to washing. This occurs in hair wherein the tips or lengths have been damaged or treated with chemical processes or exposed to external agents. In these cases, the tone may differ between root and tip. Moreover, the need for a bleaching pre-treatment before applying the direct dye considerably increases said problem of unevenness.

The purpose of the invention is to solve these three problems (resistance to washing, need for bleaching pre-treatment, and unevenness) in the field of crazy shades by using specific combinations of oxidative dyes to obtain a bright, permanent result, which is even from root to tip and applicable to hair levels 1 to 6 with no need for a bleaching pre-treatment.

### DESCRIPTION OF THE INVENTION

It has been found that the purposes described above can be achieved with a hair colouring method that uses “crazy” colors which are even and long-lasting, with no need for pre-bleaching, and which involves the use of at least one primary oxidative dye of formula I and/or II combined with a coupler of formula III:

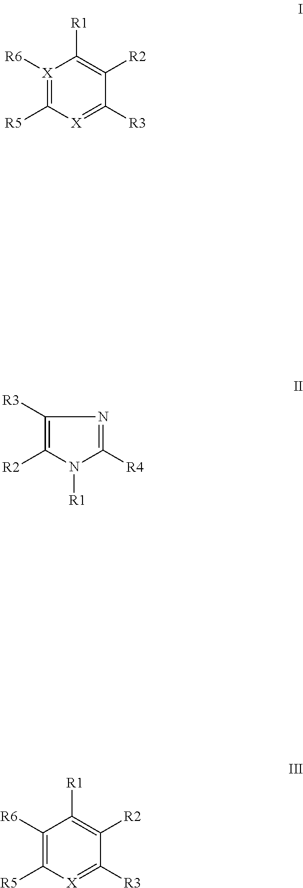

wherein:
in formula I, the X groups, which are the same or different, are a nitrogen atom or a C—R4 group; R1 is an amino group or an —N(CH₂CH₂OH)₂ group; R2, R3 and R5 are hydrogen or amino; R4 is amino;
in formula II, R1 is hydroxyethyl or hexyl, R2 and R3 are amino and R4 is hydrogen;
in formula III, X is nitrogen or C—R4, R1 is hydrogen, hydroxy, methyl; R2 is hydrogen, methyl, amino, hydroxyethylamino; R3 is hydrogen, hydroxy, amino, methyl; R5 is hydrogen, amino, hydroxy or ethyl; R6 is hydrogen, methyl, chloro or hydroxyethylamino; R4 is hydrogen or amino.

The preferred compounds of formula I have the following meanings:
X=N; R1: —NH₂; R2=—NH₂; R3=—NH₂; R5=—NH₂ (Compound A);
X=C, R1=—N(CH₂CH₂OH)₂, R2, R3, R5 and R6=—H, R4=—NH₂, (Compound F).

A preferred compound of formula II is one wherein R1 is hydroxyethyl, R2 and R3 are amino and R4 is hydrogen (Compound D).

The preferred compounds of formula III have the following meanings:
X=C, R1=—CH₃; R2 and R6=—NH—CH₂—CH₂—OH; R3, R4 and R5=—H; (Compound B);
—X=N, R1 and R2=—CH₃; R3 and R5=—OH; R6=H (Compound C);
X=C, R1=—OH; R2, R4, R5, R6=—H; R3=—NH₂; (Compound E);
X=C, R1=—OH; R2 and R6=—CH₃; R3=—NH₂; R4 and R5=—H; X=CH (Compound G);

—X=N, R1, R2 and R6=—H; R3 and R5=—NH₂; (Compound H);
X=C, R1=—OH; R2=—CH₃; R3 and R4=—H; R5=—NH₂; R6=—Cl; (Compound I);
—X=C, R1=—OH; R2=—NH₂; R3, R4 and R6=—H; R5=—CH₂CH₃; (Compound L);
—X=C, R1=—OH; R2, R5 and R6=—H; R3=—CH₃; R4=—NH₂; (Compound M).

According to one aspect of the invention, only a combination of a dye of formula I with a coupler of formula III (binary combination) is applied to the hair.

According to another aspect of the invention, only a combination of a dye of formula II with a coupler of formula III (binary combination) is applied to the hair.

According to another aspect of the invention, only a combination of a dye of formula I and a dye of formula II with a coupler of formula III (ternary combination) is applied to the hair.

The mixture of the compound of formula II, wherein R1 is hydroxyethyl, R2 and R3 are amino and R4 is hydrogen (Compound D) with the compound of formula III wherein X=N, R1 and R2=—CH3; R3 and R5=—OH; R6=H (Compound C) and with the compound of formula III wherein X=C, R1=—OH; R2, R4, R5, R6=—H; R3=—NH2 (Compound E), is novel, and is a further subject of the invention.

When the tint is mixed with the activator, which in most cases is acidic (pH about 2 to 6.5), the pH of the ready-to-use hair dye according to the invention acquires a value determined by the quantity of alkali in the tint and the quantity of acid in the oxidant, and by the mixing ratio. Depending on their composition, the ready-to-use hair dyes according to the invention can be weakly acidic, neutral or alkaline, and have a pH ranging from about 3 to 11, preferably from 6.5 to 11.

By optionally adding one part of bleaching powder to the mixture, a greater level of bleaching can be rapidly reached in a single application, giving a more intense, brighter, more even result, especially if the hair to be treated is level 1-6. The quantity of persulphate-based bleaching powder in the mixture can range between 0.1 and 20 g referred to 10 g of tint and 10 g of activator.

The composition can also include a pH adjuster selected, for example, from ammonia, monoethanolamine (MEA), 1-amino-2-propanol, 2-amino-2-methyl-propanol (AMP), 2-amino-2-methyl-1,3-propanediol, 2-amino-2-ethyl-1,3-propanediol and tris(hydroxymethyl)-aminomethane (tromethamine, Tris), sodium hydroxide, potassium hydroxide, urea, allantoin, arginine, tripotassium phosphate, sodium saccharine, triethanolamine (TEA) or combinations thereof.

The quantity of pH adjuster can range between 0.1 and 20% by weight, preferably between 0.2 and 10% by weight.

The alkalising agent can be present in free-base form or salt form.

“Activator” means hydrogen peroxide, carbamide peroxide, perborates and persulphates or peracids. The preferred compound is hydrogen peroxide. The quantity can range from 0.1 to 50%.

The hair dyes according to the invention can also contain one or more natural or synthetic additives, commonly used in solutions, creams, emulsions, gels, aerosols, foams, powders and granulates, for example solvents, such as water, low-molecular-weight aliphatic mono- or polyalcohols, esters and ethers thereof, for example alkanols, in particular having 1 to 4 carbon atoms, such as ethanol, n-propanol, isopropanol, butanol and isobutanol; bivalent or trivalent alcohols, in particular having 2 to 6 carbon atoms, such as ethylene glycol, propylene glycol, 1,3-propanediol, 1,4-butanediol, 1,5-pentanediol, 1,6-hexanediol, 1,2,6-hexanetriol, glycerin, diethylene glycol, dipropylene glycol, polyalkylene glycols, such as triethylene glycol, polyethylene glycol, tripropylene glycol and polypropylene glycol; low-molecular-weight alkyl ethers of multivalent alcohols, such as ethylene glycol monomethyl ether, ethylene glycol monoethyl ether, ethylene glycol monopropyl ether or ethylene glycol monobutyl ether, diethylene glycol monomethyl ether or diethylene glycol monoethyl ether, triethylene glycol monomethyl ether or triethylene glycol monoethyl ether; ketones and keto alcohols, in particular having 3 to 7 carbon atoms, such as acetone, methyl ethyl ketone, diethyl ketone, methyl isobutyl ketone, methyl phenyl ketone, cyclopentanone, cyclohexanone and diacetone alcohol; ethers such as dibutyl ether, tetrahydrofuran, dioxane or diisopropylether; esters such as ethyl formate, methyl formate, methyl acetate, ethyl acetate, propyl acetate, butyl acetate, phenyl acetate, ethylene glycol monoethyl ether acetate or acetic acid hydroxyethyl ester; amides such as N-methylpyrrolidone; and urea, tetramethyl urea and thiodiglycol; together with wetting agents or emulsifiers selected from anionic, cationic, non-ionogenic, amphoteric or zwitterionic agents, surfactants, such as fatty alcohol sulphates, alkylsulphonates, alkylbenzene sulphonates, alkyltrimethyl ammonium salts, alkylbetaine, α-olefin sulphonates, ethoxylated fatty alcohols, ethoxylated nonylphenols, fatty acid alkanolamines, ethoxylated esters of fatty acids, polyglycol ether sulphates of fatty acids and alkylpolyglycosides; thickeners, such as higher fatty alcohols, amide, cellulose derivatives, vaseline, paraffin oil, fatty acids and other fatty components in emulsified form, water-soluble polymer thickeners, such as natural gums, guar gum, xanthan gum, carob flour, pectin, dextran, agar-agar, amylose, amylopectin, dextrin, synthetic clays or hydrocolloids, such as polyvinyl alcohol; conditioning agents such as lanolin derivatives, cholesterol, pantothenic acid, water-soluble cationic polymers, protein derivatives, provitamins, vitamins, plant extracts, sugar and betaine; auxiliary agents such as electrolytes, antioxidants, fatty amides, sequestering agents, film-forming agents and preservatives, as well as beeswax.

The addition to the colouring preparations of non-ionic and/or anionic surfactants or emulsifiers, such as fatty alcohol sulphates, in particular lauryl sulphate or sodium cocoyl sulphate; ethoxylated fatty alcohol sulphates, in particular sodium lauryl ether sulphates with 2 to 4 molecular units of ethylene oxide, ethoxylated esters of fatty acids, ethoxylated nonylphenols, ethoxylated fatty alcohols, alkylbenzene sulphonates or alkanolamides of fatty acids, in a total quantity preferably ranging from about 0.1 to 30% by weight, more preferably from 0.2 to 15% by weight, can be particularly advantageous in this case.

Examples of useful cationic surfactants are quaternary ammonium compounds; ammonium halides such as alkyltrimethylammonium chlorides, dialkyldimethylammonium chlorides and trialkylmethylammonium chlorides. Specific examples are cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. Other useful cationic surfactants are quaternised protein hydrolysates.

As well as non-ionic organic thickeners with properties similar to wax and non-ionic surfactants, the colouring preparation can include the usual cosmetic cationic resins. Particularly preferred are Polyquaternium-6 (poly(dimethyl-diallylammonium chloride)), Polyquaternium-7 (diethyldiallylammonium chloride/acrylamide copolymer), Polyquaternium-10 (cationic cellulose), Polyquaternium-11 (diethyl sulphate of N,N-dimethylaminoethylmethacrylic acid/PVP copolymer), Polyquaternium-22, Polyquaternium-35 and Polyquaternium-37 (trimethylaminoethyl methacrylate chloride polymer), either alone or in mixtures thereof. The total quantity of said cationic resins in the hair dye can range from about 0.1 to 6% by weight.

The composition can also contain, depending on the dyes used, reducing agents and antioxidants such as sodium sulphite, sodium metabisulphite, isoascorbic acid and ascorbic acid. The composition can optionally contain direct dyes or other oxidation dyes different from those of formulas I, II and III.

Examples of additional oxidation dyes, reported with the INCI nomenclature (European Community Decision 2006/257/EC as amended—International Nomenclature of Cosmetic Ingredients), include:

1-Acetoxy-2-Methylnaphthalene, 5-Amino-4-Chloro-o-Cresol, 4-Amino-m-Cresol, 6-Amino-m-Cresol, 3-Amino-2,4-Dichlorophenol, 6-Amino-2,4-Dichloro-m-Cresol, 3-Amino-2,4-Dichlorophenol, 5-Amino-2,6-Dimethoxy-3-Hydroxypyridine, 5-Amino-2,6-Dimethoxy-3-Hydroxypyridine, 3-Amino-2,6-Dimethylphenol, 2-Amino-5-Ethylphenol, 5-Amino-4-Fluoro-2-Methylphenol Sulphate, 2-Amino-4-Hydroxyethylaminoanisole, 2-Amino-4-Hydroxyethylaminoanisole, 2-Amino-3-Hydroxypyridine, 4-Amino-2-Hydroxytoluene, 2-Aminomethyl-p-Aminophenol, 4-Amino-2-Nitrodiphenylamine-2′-Carboxylic Acid, m-Aminophenol, o-Aminophenol p-Aminophenol, 1,3-Bis-(2,4-Diaminophenoxy)propane, 4,6-Bis(2-Hydroxyethoxy)-m-Phenylenediamine, 2,6-Bis(2-Hydroxyethoxy)-3,5-Pyridinediamine, N,N-Bis(2-Hydroxyethyl)-p-Phenylenediamine, 4-Chloro-2-Aminophenol, 2-Chloro-p-Phenylenediamine, 4-Chlororesorcinol, N-Cyclopentyl-m-Aminophenol, 3,4-Diaminobenzoic Acid, 4,5-Diamino-1-((4-Chlorophenyl)Methyl)-1H-Pyrazole-Sulfate, 2,3-Diaminodihydropyrazolo Pyrazolone Dimethosulphonate, 2,4-Diaminodiphenylamine, 4,4′-Diaminodiphenylamine, 2,4-Diamino-5-Methylphenetole, 2,4-Diamino-5-Methylphenoxyethanol, 4,5-Diamino-1-Methylpyrazole, 2,4-Diaminophenol 2,4-Diaminophenoxyethanol, 2,6-Diaminopyridine, 2,6-Diamino-3-((Pyridin-3-yl)Azo)Pyridine, N,N-Diethyl-m-Aminophenol, N,N-Diethyl-p-Phenylenediamine, N,N-Diethyltoluene-2,5-Diamine, 2,6-Dihydroxy-3,4-Dimethylpyridine, 2,6-Dihydroxyethylaminotoluene, Dihydroxyindole, Dihydroxyindoline, 2,6-Dimethoxy-3,5-Pyridinediamine, m-Dimethylaminophenyl Urea, N,N-Dimethyl-p-Phenylenediamine, 2,6-Dimethyl-p-Phenylenediamine, N,N-Dimethyl 2,6-Pyridinediamine, 4-Ethoxy-m-Phenylenediamine, 3-Ethylamino-p-Cresol, 4-Fluoro-6-Methyl-m-Phenylenediamine, 1-Hexyl 4,5-Diamino Pyrazole Sulphate, Hydroquinone, Hydroxyanthraquinoneaminopropyl Methyl Morpholinium Methosulphate, Hydroxybenzomorpholine, Hydroxyethoxy Aminopyrazolopyridine, Hydroxyethylaminomethyl-p-Aminophenol, 1-Hydroxyethyl 4,5-Diamino Pyrazole, Hydroxyethyl-2,6-Dinitro-p-Anisidine, Hydroxyethyl-3,4-Methylenedioxyaniline, Hydroxyethyl-p-Phenylenediamine, 2-Hydroxyethyl Picramic Acid, 6-Hydroxyindole, Hydroxypropyl Bis(N-Hydroxyethyl-p-Phenylenediamine), Hydroxypropyl-p-Phenylenediamine, Hydroxypyridinone, Isatin, N-Isopropyl 4,5-Diamino Pyrazole, N-Methoxyethyl-p-Phenylenediamine, 6-Methoxy-2-methylamino-3-aminopyridine, 2-Methoxymethyl-p-Aminophenol, 2-Methoxymethyl-p-Phenylenediamine, 2-Methoxy-p-Phenylenediamine, 6-Methoxy-2,3-Pyridinediamine, 4-Methoxytoluene-2,5-Diamine, p-Methylaminophenol, 4-Methylbenzyl 4,5-Diamino Pyrazole, 2,2′-Methylenebis 4-Aminophenol, 3,4-Methylenedioxyaniline, 3,4-Methylenedioxyphenol, 2-Methyl-5-Hydroxyethylaminophenol, Methylimidazoliumpropyl p-Phenylenediamine, 2-Methyl-1-Naphthol, 2-Methylresorcinol, 1,5-Naphthalenediol, 1,7-Naphthalenediol, 2,3-Naphthalenediol, 2,7-Naphthalenediol, 1-Naphthol, 2-Naphthol, PEG-3 2,2′-Di-p-Phenylenediamine, p-Phenetidine, m-Phenylenediamine, p-Phenylenediamine, Phenyl Methyl Pyrazolone, N-Phenyl-p-Phenylenediamine, Picramic Acid, Pyrocatechol, Pyrogallol, Resorcinol, Sodium Picramate, Tetraaminopyrimidine, Tetrahydro-6-Nitroquinoxaline, Tetrahydropyranyl, Resorcinol, Toluene-2,5-Diamine Toluene-2,6-Diamine, Toluene-3,4-Diamine, 2,5,6-Triamino-4-Pyrimidinol, 1,2,4-Trihydroxybenzene. The oxidation dyes can be in the form of salts.

The total quantity of the combination of primary dyes and couplers according to the invention preferably ranges between about 0.001 and 20% by weight, more preferably between about 0.002 and 10% by weight, and even more preferably between about 0.01 and 6.0% by weight.

Examples of direct dyes, defined according to the INCI nomenclature (European Community Decision 2006/257/EC as amended—International Nomenclature of Cosmetic Ingredients), include:

Acid Blue 1, Acid Blue 62, Acid Brown 13, Acid Orange 3, Acid Orange 24, Acid Red 14, Acid Red 35, Acid Red 52, Acid Yellow 1,2-Amino-6-Chloro-4-Nitrophenol, 4-Amino-2-Nitrodiphenylamine-2′-Carboxylic Acid, 2-Amino-3-Nitrophenol, 2-Amino-4-Nitrophenol, 2-Amino-5-Nitrophenol, 4-Amino-2-Nitrophenol, 4-Amino-3-Nitrophenol, Basic Blue 3, Basic Blue 7, Basic Blue 9, Basic Blue 26, Basic Blue 47, Basic Blue 75, Basic Blue 99, Basic Blue 124, Basic Brown 4, Basic Brown 16, Basic Brown 17, Basic Green 1, Basic Green 4, Basic Orange 1, Basic Orange 2, Basic Orange 31, Basic Red 1, Basic Red 1:1, Basic Red 2, Basic Red 22, Basic Red 46, Basic Red 51, Basic Red 76, Basic Red 118, Basic Violet 1, Basic Violet 2, Basic Violet 3, Basic Violet 4, Basic Violet 10, Basic Violet 11:1, Basic Violet 14, Basic Violet 16, Basic Yellow 28, Basic Yellow 40, Basic Yellow 57, Basic Yellow 87, N,N′-Bis(2-Hydroxyethyl)-2-Nitro-p-Phenylenediamine, 2-Chloro-6-Ethylamino-4-Nitrophenol, 2-Chloro-5-Nitro-N-Hydroxyethyl p-Phenylenediamine, N,N′-Dimethyl-N-Hydroxyethyl-3-Nitro-p-Phenylenediamine, Direct Black 51, Direct Red 23, Direct Red 80, Direct Red 81, Direct Violet 48, Direct Yellow 12, Disperse Black 9, Disperse Blue 1, Disperse Blue 3, Disperse Blue 7, Disperse Blue 377, Disperse Brown 1, Disperse Orange 3, Disperse Red 11, Disperse Red 15, Disperse Red 17, Disperse Violet 1, Disperse Violet 4, Disperse Violet 15, HC Blue No. 2, HC Blue No. 4, HC Blue No. 5, HC Blue No. 6, HC Blue No. 8, HC Blue No. 9, HC Blue No. 10, HC Blue No. 11, HC Blue No. 12, HC Blue No. 13, HC Blue No. 14, HC Blue No. 15, HC Blue No. 16, HC Blue No. 17, HC Blue No. 18, HC Brown No. 1, HC Brown No. 2, HC Green No. 1, HC Orange No. 1, HC Orange No. 2, HC Orange No. 3, HC Orange No. 5, HC Orange No. 6, HC Red No. 1, HC Red No. 3, HC Red No. 7, HC Red No. 8, HC Red No. 9, HC Red No. 10, HC Red No. 11, HC Red No. 13, HC Red No. 14, HC Red No. 15, HC Red No. 17, HC Red No. 18, HC Violet No. 1, HC Violet No. 2, HC Yellow No. 2, HC Yellow No. 4, HC Yellow No. 5, HC Yellow No. 6, HC Yellow No. 7, HC Yellow No. 8, HC Yellow No. 9, HC Yellow No. 10, HC Yellow No. 11, HC Yellow No. 12, HC Yellow No. 13, HC Yellow No. 14, HC Yellow No. 15, HC Yellow No. 16, HC Yellow No. 17, 2-Hydroxyethylamino-5-Nitroanisole, Hydroxyethyl-2-Nitro-p-Toluidine, 4-Hydroxypropylamino-3-Nitrophenol, 3-Methylamino-4-Nitrophenoxyethanol, 3-Nitro-4-Aminophenoxyethanol, 3-Nitro-p-Cresol, 2-Nitro-5-Glyceryl Methylaniline, 4-Nitroguaiacol, 3-Nitro-p-Hydroxyethylaminophenol, 2-Nitro-N-Hydroxyethyl-p-Anisidine, Nitrophenol, 4-Nitrophenyl Aminoethylurea, 4-Nitro-o-Phenylenediamine, 4-Nitro-m-Phenylenediamine, 4-Nitro-o-Phenylenediamine, 2-Nitro-p-Phenylenediamine, 6-Nitro-2,5-Pyridinediamine, 6-Nitro-o-Toluidine, Pigment Blue 15, Pigment Blue 15:1, Pigment Violet 23, Pigment Yellow 13, Solvent Black 3, Solvent Black 5, Solvent Blue 35, Solvent Yellow 85, Solvent Yellow 172, Tetrabromophenol Blue, Tetrahydro-6-Nitroquinoxaline, Tetrahydropyranyl Resorcinol.

For the use of oxidative hair tints, the hair dyes are mixed with an oxidative solution immediately before dyeing the hair, and a sufficient quantity of ready-to-use hair colouring mixture, generally about 60 to 200 grams, depending on the thickness and quantity of the hair, is applied.

The mixture is left on the hair for 2 to 60 minutes at a temperature of 5 to 50° C.; the hair is then rinsed with water and dried. If necessary, the hair is washed with shampoo after rinsing and optionally rinsed again with a weak organic acid, such as an aqueous solution of tartaric acid. The hair is then dried.

The hair colouring preparation according to the invention gives an intense, protective, delicate hair color. The hair can be dyed from the undamaged root to severely damaged tips.

The color of the cream during oxidation is the same as the color obtained on the hair.

### EXAMPLES

The preferred compounds of formula I, II and III, indicated above by the letters A-M, are used.

The data set out in Tables 1 and 2 demonstrate that simple binary combinations of said preferred dyes produce surprisingly bright “crazy” colors on level 6 natural hair without pre-bleaching.

TABLE 1
color result from level 6 after 50 minutes application

| PRIMARY DYE | DYE COUPLER | ACTI- | COLOUR RESULT |
|---|---|---|---|
| (% dye) | (% dye) | VATOR | from level 6 |
| A (2.15) | B (1.898) | 40 V | BRIGHT RED |
| A (1.082) | C (0.632) | 40 V | BRIGHT YELLOW |
| D (1.531) | E (0.04) | 40 V | BRIGHT ORANGE |
| | C (0.836) | | |
| F (1.407) | G (0.618) | 40 V | BRIGHT VIOLET |
| D (0.825) | H (0.375) | 40 V | BRIGHT MAGENTA |
| F (1.406) | I (0.71) | 40 V | BRIGHT BLUE |
| F (1.073) | H (0.375) | 40 V | BRIGHT BLUE-GREEN |
| D (1.083) | L (0.783) | 40 V | BRIGHT RED |
| M (0.555) | L (0.783) | 40 V | YELLOW |
| F (0.493) A (0.05) | B (0.18) | 40 V | BRIGHT BLUE- |
| | C (0.13) | | VIOLET |
| D (1.084) | B (0.949) | 40 V | BRIGHT PURPLE |
| F (0.385) | H (0.1211) | 40 VOL | BRIGHT AZURE |
| | B (0.026) | | |

A more intense result can be obtained by combining the activator with a bleaching powder containing persulphates (EQUIPMENT SUPERMECHES HIGH LIFT by ALFAPARF) with a dilution ratio of 1:1:0.5 (dye:activator:bleaching powder).

TABLE 2
color result from level 6 with 50 minutes application combined with
bleaching powder.

| | DYE | ACTIVATOR + | |
|---|---|---|---|
| PRIMARY DYE | COUPLER | BLEACHING | COLOUR RESULT |
| (% dye) | (% dye) | POWDER | from level 6 |
| A (2.15) | B (1.898) | 40 V + persulphates | INTENSE BRIGHT |
| | | | RED |
| A (1.082) | C (0.632) | 40 V + persulphates | INTENSE BRIGHT |
| | | | YELLOW |
| D (1.531) | E (0.04) | 40 V + persulphates | INTENSE BRIGHT |
| | C (0.836) | | ORANGE |
| F (1.407) | G (0.618) | 40 V + persulphates | INTENSE BRIGHT |
| | | | VIOLET |
| D (0.825) | H (0.375) | 40 V + persulphates | INTENSE BRIGHT |
| | | | MAGENTA |
| F (1.406) | I (0.71) | 40 V + persulphates | INTENSE BRIGHT |
| | | | BLUE |
| F (1.073) | H (0.375) | 40 V + persulphates | INTENSE BRIGHT |
| | | | BLUE-GREEN |
| D (1.083) | L (0.783) | 40 V + persulphates | INTENSE BRIGHT |
| | | | RED |
| M (0.555) | L (0.783) | 40 V + persulphates | INTENSE |
| | | | YELLOW |
| F (0.493) A (0.05) | B (0.18) | 40V +persulphates | INTENSE BRIGHT |
| | C (0.13) | | BLUE-VIOLET |
| D (1.084) | B (0.949) | 40V + persulphates | INTENSE BRIGHT |
| | | | PURPLE |
| F (0.385) | H (0.1211) | 40 VOL + | INTENSE BRIGHT |
| | B (0.026) | persulphates | AZURE |

Table 3 shows the results obtainable on bleached hair in case the hairdresser prefers to follow this method of application.

TABLE 3
color result after bleaching, with processing time of 15 minutes

| | DYE | | |
|---|---|---|---|
| PRIMARY DYE | COUPLER | ACTIVATOR | COLOUR RESULT ON |
| (% dye) | (% dye) | (V = volume) | LOCK after bleaching |
| A (2.15) | B (1.898) | 10 V | BRIGHT RED |
| A (1.082) | C (0.632) | 10 V | BRIGHT YELLOW |
| D (1.531) | E (0.04) | 10 V | BRIGHT ORANGE |
| | C (0.836) | | |
| F (1.407) | G (0.618) | 10 V | BRIGHT VIOLET |
| D (0.825) | H (0.375) | 10 V | BRIGHT MAGENTA |
| F (1.406) | I (0.71) | 10 V | BRIGHT BLUE |
| F (1.073) | H (0.375) | 10 V | BRIGHT BLUE-GREEN |
| D (1.083) | L (0.783) | 10 V | BRIGHT RED |
| M (0.555) | L (0.783) | 10 V | YELLOW |
| F (0.493) | B (0.18) | 10 V | BRIGHT BLUE-VIOLET |
| A (0.05) | C (0.13) | | |
| D (1.084) | B (0.949) | 10 V | BRIGHT PURPLE |
| F (0.385) | H (0.1211) | 10 V | BRIGHT AZURE |
| | B (0.026) | | |

The combinations described herein give “crazy” colors very similar to those obtained with a direct dye (in terms of both tone and brightness), but with improved resistance to washing.

The data in Table 4 demonstrate that the combination of A and B resists washing better than a direct dye that gives the same tone.

TABLE 4
Colour fading after 6 washes for a composition according to the
invention vs. a direct dye.

| Composition | % color fading after 6 washes |
|---|---|
| A + B | 10% |
| Basic red 51 (1%) | 35% |

The data in Table 5 demonstrate that the compositions according to the invention, applied to a model, give greater evenness from root to tip than the direct dye compositions reported in Table 6.

TABLE 5
Root-to-tip evenness value for the compositions according to the
invention (visual evaluation).

| PRIMARY DYE | DYE COUPLER | ACTIVATOR | ROOT-TO-TIP |
|---|---|---|---|
| (% dye) | (% dye) | (V = volume) | EVENNESS |
| A (2.15) | B (1.898) | 10 V | HIGH |
| A (1.082) | C (0.632) | 10 V | HIGH |
| D (1.531) | E (0.04)C (0.836) | 10 V | HIGH |
| F (1.407) | G (0.618) | 10 V | HIGH |
| D (0.825) | H (0.375) | 10 V | HIGH |
| F (1.406) | I (0.71) | 10 V | HIGH |
| F (1.073) | H (0.375) | 10 V | HIGH |
| D (1.083) | L (0.783) | 10 V | HIGH |
| M (0.555) | L (0.783) | 10 V | HIGH |
| F (0.493) A (0.05) | B (0.18) C (0.13) | 10 V | HIGH |
| D (1.084) | B (0.949) | 10 V | HIGH |
| F (0.385) | H (0.1211) B (0.026) | 10 V | HIGH |

TABLE 6

| DYE (% dye) | ROOT-TO-TIP EVENNESS |
|---|---|
| BASIC RED 51 (0.5) + | MODERATE |
| BASIC YELLOW 87 (0.5) | |
| HC BLUE 15 (0.5) | LOW |

The dye combinations were inserted in the cosmetic form shown in Table 7.

TABLE 7
Cosmetic base used for the various combinations of oxidative dyes

| INGREDIENT NAME | % QUANTITY |
|---|---|
| AQUA (WATER) | Q.s. to 100 |
| CETEARYL ALCOHOL | 6 |
| DYES | 0.001 to 20% |
| STEARYL ALCOHOL | 6 |
| PROPYLENE GLYCOL | 4 |
| ETHANOLAMINE | 3 |
| ETHOXYDIGLYCOL | 2 |
| AMMONIA | 1.5 |
| CETEARETH-50 | 2 |
| LAURYL ALCOHOL | 2 |
| PEG-40 HYDROGENATED CASTOR OIL | 2 |
| MYRISTYL ALCOHOL | 0.5 |
| COCAMIDOPROPYL BETAINE | 0.5 |
| PARFUM (FRAGRANCE) | 0.5 |
| SODIUM SULPHITE | 0.5 |
| GLYCERYL STEARATE SE | 0.4 |
| POLYQUATERNIUM-22 | 0.2 |
| DIPROPYLENE GLYCOL | 0.2 |
| ERYTHORBIC ACID | 0.3 |
| EDTA | 0.2 |

1. A method for colouring hair with even, long-lasting crazy colors with no need for pre-bleaching, which comprises applying to the hair at least one primary oxidative dye of formula I and/or II in combination with a coupler of formula III: 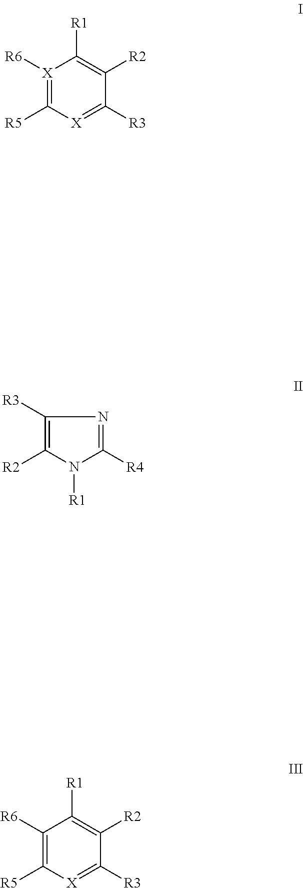
wherein:
in formula I, the X groups, which can be the same or different, are a nitrogen atom or a C—R4 group; R1 is an amino group or a —N(CH₂CH₂OH)₂ group, R2, R3, R5 are hydrogen or amino; R4 is amino;
in formula II, R1 is hydroxyethyl or hexyl, R2 and R3 are amino and R4 is hydrogen;
in formula III, X is nitrogen or C—R4, R1 is hydrogen, hydroxy, methyl; R2 is hydrogen, methyl, amino, hydroxyethylamino; R3 is hydrogen, hydroxy, amino, methyl; R5 is hydrogen, amino, hydroxy or ethyl; R6 is hydrogen, methyl, chlorine or hydroxyethylamino; R4 is hydrogen or amino.

2. The method according to claim 1 wherein only a dye of formula I and a coupler of formula III are applied to the hair.

3. The method according to claim 1 wherein only a dye of formula II and a coupler of formula III are applied to the hair.

4. The method according to claim 1 wherein only a dye of formula I, a dye of formula II and a coupler of formula III are applied to the hair.

5. A method according to claim 1 wherein the compounds of formula I have the following meanings:
—X=N; R1: —NH₂; R2=—NH₂; R3=—NH₂; R5=—NH₂
—X=C, R1=—N(CH₂CH₂OH)₂, R2, R3, R5 and R6=—H R4=—NH₂.

6. A method according to claim 1, wherein the compounds of formula III have the following meanings:
X=C, R1=—CH₃; R2 and R6=—NH—CH₂—CH₂—OH; R3, R4 and R5=—H;
X=N, R1 and R2=—CH₃; R3 and R5=—OH; R6=H
X=C, R1=—OH; R2, R4, R5, R6=—H; R3=—NH₂;
X=C, R1=—OH; R2 and R6=—CH₃; R3=—NH₂; R4 and R5=—H; X=CH;
X=N, R1, R2 and R6=—H; R3 and R5=—NH₂;
X=C, R1=—OH; R2=—CH₃; R3 and R4=—H; R5=—NH₂; R6=—Cl;
X=C, R1=—OH; R2=—NH₂; R3, R4 and R6=—H; R5=—CH₂CH₃;
X=C, R1=—OH; R2, R5 and R6=—H; R3=—CH₃; R4=—NH₂.

7. A method according to claim 1 wherein the compounds of formula I, II and III are mixed with an activator and applied directly to the hair with no need for a bleaching pre-treatment.

8. A method according to claim 7, wherein said activator is hydrogen peroxide.

9. A method according to claim 1 wherein persulphates are added to the dye mixture so as to obtain, after a single application, more marked bleaching and therefore a more intense, even color result.

10. A composition for colouring hair comprising a mixture of primary oxidative dyes/couplers consisting of a compound of formula II wherein R1 is hydroxyethyl, R2 and R3 are amino and R4 is hydrogen, a compound of formula III wherein X=N, R1 and R2=—CH3; R3 and R5=—OH, R6=H, and a compound of formula III wherein X=C, R1=—OH; R2, R4, R5, R6=—H; R3=—NH2.
